(19) United States
(12) Patent Application Publication
(10) Pub. No.: US 2021/0139493 A1
(43) Pub. Date: May 13, 2021

(54) PREPARATION OF VELPATASVIR AND DERIVATIVE THEREOF

(71) Applicant: SHANGHAI FOREFRONT PHARMACEUTICAL CO., LTD, Shanghai (CN)

(72) Inventors: Shaojun FU, Shanghai (CN); Chengjun HUANG, Shanghai (CN); Yi REN, Shanghai (CN); Wei LI, Shanghai (CN)

(21) Appl. No.: 16/488,844

(22) PCT Filed: Feb. 27, 2018
(86) PCT No.: PCT/CN2018/077438

§ 371 Date: Aug. 26, 2019

(30) Foreign Application Priority Data
Feb. 27, 2017 (CN) 201710109238.1

(51) Int. Cl. C07D 491/052; C07D 405/12; C07D 405/14

(52) U.S. Cl. CPC C07D 491/052; C07D 405/14; C07D 405/12

(57) ABSTRACT

The present invention relates to a preparation method for velpatasvir and a derivative thereof. Specifically, in the present invention, velpatasvir and the derivative thereof is prepared by means of an intermediate compound represented by the following formula (definitions of the groups are described in the specifications). By means of the method, byproducts are fewer, costs are low, and the method is applicable to industrial production of velpatasvir.

[chemical structure]

### FIELD OF THE INVENTION

The invention relates to the field of drug synthesis, and in particular, the invention provides an intermediate of an anti-hepatitis drug, velpatasvir (GS-5816) and a preparation method thereof.

### BACKGROUND OF THE INVENTION

Velpatasvir (GS-5816) is a new generation of NS5A inhibitor developed by Gilead Science Co., Ltd. The combination of Velpatasvir and Sofosbuvir will be the first single-tablet oral regimen for the treatment of pan-genotype hepatitis C, which is effective for all six genotypes. The structure of velpatasvir is as follows:

[chemical structure]

The compound represented by Formula 1 is an important intermediate for the preparation of velpatasvir (GS-5816). Currently, the known preparation method of the compound of formula 1 is mainly the synthetic route reported in WO2013075029, and the three routes are listed as follows respectively:

Route 1

[chemical structure]

Route 2:

[chemical structure]

Route 3:

[chemical structure]

Among them, it is necessary to carry out the ring-closing reaction of two imidazole rings at one time at a high temperature for the synthetic route 1 and 3, which lead to many by-products and generate a large amount of tar, thus making the later purification quite difficult (which need to remove tar by means of column chromatography and the like), and difficult for industrialization. There is a three-step palladium-catalyzed coupling reaction in the synthesis route 2 reaction, which makes the preparation of the velpatasvir by this route of very high cost and relatively much side reaction, thus making the later purification quite difficult.

In summary, there is a need for a method for preparing an intermediate of the formula 1 which is of low cost, convenient in purification, and suitable for industrial production.

### SUMMARY OF THE INVENTION

The object of the present invention is to provide a method for preparing an intermediate of formula 1 which is of low cost, convenient in purification, and suitable for industrial production.

According to a first aspect of the present invention, a method for preparing an intermediate compound of velpatasvir represented by formula 1 is provided,

[chemical structure]

wherein it comprises the steps:
(1) condensing a compound of formula 7 and a compound of formula 6 to provide a compound of formula 5;

[chemical structure]

(2) subjecting the compound of formula 5 to a cyclization reaction to prepare a compound of formula 4;

[chemical structure]

(3) reacting the compound of formula 4 with a substitution reagent to prepare a compound of formula 3

[chemical structure]

(4) condensing the compound of formula 3 with a compound of formula 8 to obtain a compound of formula 2;

[chemical structure]

(5) subjecting the compound of formula 2 to a cyclization reaction to give a compound of formula 1;

[chemical structure]

in the above formulas,
Y is selected from the group consisting of —F, —Cl, —Br, —I, —OTs, —OSO₂CF₃, —SO₂R₆, —OP(O)(OR₆)₂; wherein R₆ is C1-C10 alkane group, C6-C10 aromatic hydrocarbon group or substituted aromatic hydrocarbon group; preferably, said Y is selected from the group consisting of: —Cl, —Br, —I;
Z is selected from the group consisting of H, alkali metal ions, alkaline earth metal ions; preferably, said Z is selected from the group consisting of: —H, —Na, —K, —Li, —Cs;
R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
R₂ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl, alkoxymethyl, alkylsulfonyl, substituted arylsulfonyl, L-methoxycarbonylvalyl;
wherein said “substituted” means that a group is substituted by one or more substituents selected from the group consisting of halogen, C1-C6 alkyl, C2-C10 acyl.

In another preferred embodiment, when Z is H, the steps (1) and (4) are carried out under alkali condition.

In another preferred embodiment, in the step (2) and/or the step (5), the cyclization reaction is carried out in the presence of ammonia or a derivative thereof selected from the group consisting of C1-C6 ammonium carboxylate, ammonium salt of inorganic acid, carbamide, NH₃, methylsilicone diamine, or the combinations thereof, preferably ammonium acetate, ammonium formate, ammonium hydrogencarbonate, or the combinations thereof.

In another preferred embodiment, in the step (2), the reaction temperature is between 70 to 120° C., preferably between 80 to 100° C.

In another preferred embodiment, in the step (2), the molar ratio of the compound of the formula 5 to the cyclizing reagent is from 1:1 to 10, preferably from 1:3 to 7.

In another preferred embodiment, in the step (5), the reaction temperature is between 70 to 140° C., preferably between 80 to 100° C.

In another preferred embodiment, in the step (5), the molar ratio of the compound of the formula 2 to the cyclizing reagent is 1:10-40, preferably 1:15-30, more preferably 1:18-25.

In another preferred embodiment, in the step (4), the alkaline condition is provided by adding an alkaline agent selected from the group consisting of alkali metal hydrogencarbonate, alkaline earth metal hydrogencarbonate, alkali metal carbonate, alkaline earth metal carbonate, alkali metal hydroxide, alkaline earth metal hydroxide, alkali metal phosphate, alkaline earth metal phosphate, alkali metal hydrogen phosphate salt, alkaline earth metal hydrogen phosphate salt, DBU, DBN,

[chemical structure]

or a combination thereof, wherein R₃, R₄, R₅ are each independently H or a C1-C4 alkane; preferably, the alkaline agent is selected from the group consisting of potassium carbonate, cesium carbonate, or the combination thereof.

In another preferred embodiment, in the step (4), the reaction temperature is from 0 to 100° C., preferably from 0 to 70° C., more preferably from 20 to 60° C.

In another preferred embodiment, in the step (4), the molar ratio of the compound 3 to the compound 8 is 1:0.5-2.

In another preferred embodiment, in the step (1), the alkaline agent includes, but is not limited to, an alkali metal or alkaline earth metal hydrogencarbonate, alkali metal or an alkaline earth carbonate, alkali metal or alkaline earth metal hydroxide, alkali metal or alkaline earth metal phosphate, alkali metal or alkaline earth metal hydrogen phosphate, DBU, DBN,

[chemical structure]

wherein R₃, R₄, and R₅ are each independently H or C1-C4 alkane; preferably potassium carbonate, cesium carbonate.

In another preferred embodiment, in the step (1), the molar ratio of the compound of the formula 7 to the compound of the formula 6 is 1:0.5-2.

In another preferred embodiment, in the step (1), the molar ratio of the compound of the formula 7 to the alkaline agent is 1:1-5.

In another preferred embodiment, in the step (1), the reaction temperature is from 0 to 100° C., and the comparatively suitable temperature is from 20 to 70° C., preferably from 25 to 50° C.

In another preferred embodiment, in the step (3), the substitution reagent is halogenating reagent, preferably halogenating reagent selected from the group consisting of N-halogenated succinimide, 5,5-dimethyl-1,3 dihalohydantoin, halogen, chlorinated halide, lithium halide, sodium halide, potassium halide, pyridine trihalide, trihalide tetrabutyl ammonium, trihalide trimethylammonium, trihalide triethylammonium, sodium hypohalite, potassium hypohalite, lithium hypohalite, sodium sulfite, potassium sulfite, sodium sulfite, or a combination thereof, preferred is N-bromosuccinimide (NBS), 5,5-dimethyl-1,3-dibromohydantoin, pyridine tribromide, or the combinations thereof.

In another preferred embodiment, in the step (3), the molar ratio of the compound of the formula 4 to the halogenating agent is 1:0.5-2.

In the second aspect of the present invention, an intermediate compound of the following formula is provided:

[chemical structure]

wherein, R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
X is selected from the group consisting of —H, —F, —Cl, —Br, —I, —OTs, —OSO₂CF₃, —SO₂R₆, —OP(O)(OR₆)₂,

[chemical structure]

wherein R₆ is C1-C10 alkyl, C6-C10 aromatic hydrocarbon group or substituted aromatic hydrocarbon group; preferably, the compound is selected from the group consisting of:

[chemical structure]

wherein, Y is selected from the group consisting of —F, —Cl, —Br, —I, —OTs, —OSO₂CF₃, —SO₂R₆, —OP(O)(OR₆)₂; wherein R₆ is C1-C10 alkyl, C6-C10 aromatic hydrocarbon group or substituted aromatic hydrocarbon group.

In the third aspect of the present invention, an intermediate compound of the following formula is provided:

[chemical structure]

In the fourth aspect of the present invention, a method for the preparation of an intermediate compound of velpatasvir of formula 1 is provided, wherein the method comprises the steps:

Subjecting a compound of formula 2 to a cyclization reaction to give a compound of formula 1;

[chemical structure]

in the above formulas,
R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
R₂ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl, alkoxymethyl, alkylsulfonyl, substituted arylsulfonyl, L-methoxycarbonylvalyl;
wherein said “substituted” means that a group is substituted by one or more substituents selected from the group consisting of halogen, C1-C6 alkyl, C2-C10 acyl.

In another preferred embodiment, the method further comprises the following steps:
under an alkali condition, condensing the compound of formula 3 with compound of formula 8 to obtain a compound of formula 2;

[chemical structure]

wherein,
Y is selected from the group consisting of —F, —Cl, —Br, —I, —OTs, —OCF₂CF₃, —SiO₂R₆, —OP(O)(OR₆)₂; wherein R₆ is C1-C10 alkane group, C6-C10 aromatic hydrocarbon group or substituted aromatic hydrocarbon group; preferably, said Y is selected from the group consisting of: —Cl, —Br, —I;
Z is selected from the group consisting of H, alkali metal ions, alkaline earth metal ions; preferably, said Z is selected from the group consisting of: —H, —Na, —K, —Li, —Cs.

In another preferred embodiment, when Z is H, the steps (1) and (4) are carried out under an alkali condition.

In the fifth aspect of the present invention, a method for the preparation compound velpatasvir is provided, wherein the method comprises the steps:

Subjecting a compound of formula 2 to a cyclization reaction to give a compound of formula 1;

[chemical structure]

and
preparing verapitavir with the compound of formula 1;
in the above formulas,
R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
R₂ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl, alkoxymethyl, alkylsulfonyl, substituted arylsulfonyl, L-methoxycarbonylvalyl;
wherein said “substituted” means that a group is substituted by one or more substituents selected from the group consisting of halogen, C1-C6 alkyl, C2-C10 acyl.

In another preferred embodiment, the method comprises the following steps:

[chemical structure]

deprotectin the compound of formula 1 to give a compound of formula VLP-1;

[chemical structure]

condensing reaction formula VLP-1 compound with D-phenylglycine to give velpatasvir (VLP).

It should be understood that, in the present invention, each of the technical features specifically described above and below (such as those in the Examples) can be combined with each other, thereby constituting new or preferred technical solutions which need not be specified again herein.

### EMBODIMENTS FOR CARRYING OUT THE INVENTION

Based on long-term and in-depth research, the present inventors have provided a method for preparing an intermediate compound of velpatasvir of formula 1 which can solve the problems of the prior art, such as high cost, too many reaction by-products, and difficulty in later purification. The preparation method has advantages such as of low cost, convenience in purification and being suitable for industrial production. The present invention is completed on this basis.

Preparation of Compound of Formula 1

The inventor achieved the purpose of the present invention by the following technical solution (shown in Route 4):

[chemical structure]

The method of the invention comprises condensing a compound of formula 7 and a compound of formula 6 under alkali conditions to obtain a compound of formula 5; subjecting the compound of formula 5 to a cyclization reaction with ammonia or a derivative thereof at a high temperature to prepare a compound of formula 4; reacting the compound of formula 4 with a substitution reagent to carry out carbonyl α-position substitution so as to obtain a compound of formula 3; condensing the compound of formula 3 with a compound of formula 8 under an alkali condition to obtain a compound of formula 2; subjecting the compound of formula 2 to a cyclization reaction with ammonia or a derivative thereof at a high temperature to prepare a compound of formula 1; oxidizing or oxidizing-deprotecting the compound of formula 1 to give VLP-1, and condensing VLP-1 with D-phenylglycine to give velpatasvir VLP (GS-5816).

Specifically, the method of the present invention includes the following steps:
(1) under an alkali condition, condensing a compound of formula 7 and a compound of formula 6 to provide a compound of formula 5;

[chemical structure]

(2) subjecting the compound of formula 5 to a cyclization reaction to prepare a compound of formula 4;

[chemical structure]

(3) reacting the compound of formula 4 with a substitution reagent to prepare a compound of formula 3;

[chemical structure]

(4) under an alkali condition, condensing the compound of formula 3 with a compound of formula 8 to obtain a compound of formula 2

[chemical structure]

(5) subjecting the compound of formula 2 to a cyclization reaction to give a

[chemical structure]

in the above formulas,
Y is selected from the group consisting of —F, —Cl, —Br, —I, —OTs, —OSO₂CF₃, —SO₂R₆, —OP(O)(OR₆)₂; wherein R₆ is C1-C10 alkane group, C6-C10 aromatic hydrocarbon group or substituted aromatic hydrocarbon group; preferably, said Y is selected from the group consisting of: —Cl, —Br, —I;
R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
R₂ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl, alkoxymethyl, alkylsulfonyl, substituted arylsulfonyl, L-methoxycarbonylvalyl;
wherein said “substituted” means that a group is substituted by one or more substituents selected from the group consisting of halogen, C1-C6 alkyl, C2-C10 acyl.

In another preferred embodiment, in the step (5), the reaction temperature is between 70 to 140° C., preferably between 80 to 100° C.

In another preferred embodiment, in the step (2) and/or the step (5), the cyclization reaction is carried out in the presence of ammonia or a derivative thereof selected from the group consisting of C1-C6 ammonium carboxylate, ammonium salt of inorganic acid, carbamide, NH₃, methylsilicone diamine, or the combinations thereof, preferably ammonium acetate, ammonium formate, ammonium hydrogencarbonate, or the combinations thereof.

In another preferred embodiment, in the step (4), the alkaline condition is provided by adding an alkaline agent selected from the group consisting of alkali metal hydrogencarbonate, alkaline earth metal hydrogencarbonate, alkali metal carbonate, alkaline earth metal carbonate, alkali metal hydroxide, alkaline earth metal hydroxide, alkali metal phosphate, alkaline earth metal phosphate, alkali metal hydrogen phosphate salt, alkaline earth metal hydrogen phosphate salt, DBU, DBN,

[chemical structure]

or a combination thereof, wherein R₃, R₄, R₅ are each independently H or a C1-C4 alkane; preferably, the alkaline agent is selected from the group consisting of potassium carbonate, cesium carbonate, or the combination thereof.

In another preferred embodiment, in the step (4), the reaction temperature is from 0 to 100° C., preferably from 0 to 70° C., more preferably from 20 to 60° C.

In another preferred embodiment, in the step (1), the reaction temperature is from 0 to 100° C., and a relatively suitable temperature is from 20 to 70° C., preferably from 25 to 50° C.

In another preferred embodiment, in the step (1), the alkaline agent includes, but is not limited to, an alkali metal or alkaline earth metal hydrogencarbonate, alkali metal or an alkaline earth carbonate, alkali metal or an alkaline earth hydroxide, alkali metal or alkaline earth metal phosphate alkali metal or alkaline earth metal hydroxide phosphate, DBU, DBN,

[chemical structure]

wherein R₃, R₄, and R₅ are each independently H or C1-C4 alkane; preferably potassium carbonate, cesium carbonate.

In another preferred embodiment, in the step (2), the reaction temperature is between 70 to 120° C., preferably between 80 to 100° C.

In another preferred embodiment, in the step (3), the substitution reagent is halogenating reagent, preferably halogenating reagent selected from the group consisting of N-halogenated succinimide, 5,5-dimethyl-1,3 dihalohydantoin, halogen, chlorinated halide, lithium halide, sodium halide, potassium halide, pyridine trihalide, trihalide tetrabutyl ammonium, trihalide trimethylammonium, trihalide triethylammonium, sodium hypohalite, potassium hypohalite, lithium hypohalite, sodium sulfite, potassium sulfite, sodium sulfite, or a combination thereof, preferred is N-bromosuccinimide (NBS), 5,5-dimethyl-1,3-dibromohydantoin, pyridine tribromide, or the combinations thereof.

Velpatasvir Intermediate Compound

The present invention also provides an intermediate compound as shown in the following formula for preparing velpatasvir:

[chemical structure]

wherein, R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
X is selected from the group consisting of —H, —F, —Cl, —Br, —I, —OTs, —OSO₂CF₃, —SO₂R, —OP(O)(OR₆)₂,

[chemical structure]

wherein R₆ is C1-C10 alkyl, C6-C10 aromatic hydrocarbon group or substituted aromatic hydrocarbon group.

In another preferred embodiment, the compound is selected from the following group:

[chemical structure]

wherein, Y is selected from the group consisting of —F, —Cl, —Br, —I, —OTs, —OSO₂CF₃, —SO₂R₆, —OP(O)(OR₆)₂; wherein R is C1-C10 alkyl, C6-C10 aromatic hydrocarbon group or substituted aromatic hydrocarbon group.

Compared with the prior art, the main advantages of the present invention includes:
(1) The vertapavir treatment route of the invention produces few by-products, so that it is easy to purify vertapavir, and it is suitable for industrial preparation of voratavivir;
(2) The method of the present invention does not require the use of expensive reagents, and therefore is of low cost, thus being suitable for mass production.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacturer's instructions. Unless indicated otherwise, parts and percentage are calculated by weight.

### Example 1: Synthesis of Compound 5a

[chemical structure]

In a 100 ml reaction flask, 3.7 g of compound 7, 2.85 g of compound 6a, 2.76 g of potassium carbonate, and 55.5 ml of dichloromethane were added, heated to 30-35° C., and stirred for 16 hours. The reaction raw material 7 was consumed under LC monitoring, and then the reaction was quenched by addition of water. The reaction mixture was washed with water, dried over sodium sulfate, and concentrated to give compound 5a (5.68 g, purity 96.58%, yield 100%).

### Example 2: Synthesis of Compound 5b

[chemical structure]

In a 100 ml reaction flask, 3.7 g of compound 7, 3.3 g of compound 6b, 2.76 g of potassium carbonate, and 55.5 of ml dichloromethane were added, heated to 30-35° C., and stirred for 16 hours. The reaction raw material 7 was consumed under LC monitoring, and then the reaction was quenched by addition of water. The reaction mixture was washed with water, dried over sodium sulfate and evaporated to provide compound 5b (5.45 g, yield 98%).

### Example 3: Synthesis of Compound 4a

[chemical structure]

In a 100 ml reaction flask, 2.0 g of compound 5a, 2.0 g of ammonium acetate, 2.5 ml of ethylene glycol methyl ether, 25 ml of toluene were added, and stirred at 90° C. for 16 hours. The reaction raw material 5a was consumed under LC monitoring. The reaction mixture was washed with 10 ml of hot water and saturated brine, then dried over anhydrous sodium sulfate, and evaporated to give compound 4a (1.97 g, purity 93.45%, yield 95.5%).

¹H NMR (400 MHz, Chloroform) 8.27 (s, 1H), 8.09 (s, 1H), 7.99 (s, 1H), 7.90 (s, 1H), 7.62 (s, 1H), 7.35 (s, 1H), 5.35 (s, 1H), 5.29 (s, 2H), 4.46 (s, 1H), 3.35 (d, J=4.4 Hz, 4H), 3.20 (s, 1H), 2.99 (d, J=16.8 Hz, 3H), 2.66 (s, 1H), 2.51 (s, 1H), 2.32 (s, 1H), 2.14 (s, 1H), 1.75 (s, 1H), 1.42 (s, 9H).

### Example 4: Synthesis of Compound 4b

[chemical structure]

In a 100 ml reaction flask, 2.0 g of compound 5b, 2.2 g of ammonium acetate, 2.5 ml of ethylene glycol methyl ether, and 25 ml of toluene were added, and stirred at 90° C. for 16 hours. The reaction raw material 5b was consumed by LC monitoring. The reaction mixture was washed with 10 ml of hot water and saturated brine, then dried over anhydrous sodium sulfate, and evaporated to give compound 4b (1.6 g, yield: 84.5%).

¹H NMR (400 MHz, Chloroform) 8.27 (s, 1H), 8.09 (s, 1H), 7.99 (s, 1H), 7.90 (s, 1H), 7.62 (s, 1H), 7.33 (t, J=6.0 Hz, 6H), 5.30 (s, 1H), 5.21 (s, 2H), 5.02 (s, 2H), 4.49 (s, 1H), 3.35 (d, J=9.4 Hz, 4H), 3.20 (s, 1H), 3.00 (d, J=20.2 Hz, 3H), 2.94-2.75 (m, 1H), 2.66 (s, 1H), 2.50 (s, 1H), 2.35 (s, 1H), 2.14 (s, 1H), 1.77 (s, 1H).

### Example 5: Synthesis of Compound 3a

[chemical structure]

In a 100 ml reaction flask, 2.65 g of compound 4a, 1.74 g of pyridine tribromide compound, 26.5 ml of dichloromethane, 3.0 ml of methanol were added, and stirred at 20-25° C. for 3 hours. The reaction raw material 4a was consumed under LC monitoring. 10 ml of water was added and stirred for 10 min. The mixture was partitioned, and the organic layer was washed twice with water, dried over anhydrous sodium sulfate, and concentrated to give compound 3a (1.96 g, yield 84%).

¹H NMR (400 MHz, Chloroform) δ 8.27 (s, 1H), 8.09 (s, 1H), 7.99 (s, 1H), 7.90 (s, 1H), 7.62 (s, 1H), 7.35 (s, 1H), 5.49 (s, 1H), 5.31 (d, J=17.2 Hz, 3H), 4.47 (s, 1H), 3.35 (d, J=4.7 Hz, 4H), 3.20 (s, 1H), 3.02 (s, 1H), 2.83 (d, J=13.0 Hz, 2H), 2.75 (s, 1H), 2.57 (s, 1H), 2.51 (s, 1H), 2.32 (s, 1H), 1.75 (s, 1H), 1.42 (s, 9H).

### Example 6: Synthesis of Compound 3b

[chemical structure]

In a 100 ml reaction flask, 2.65 g of compound 4b, 1.65 of g pyridine tribromo compound, 26.5 ml of dichloromethane, 3.0 ml of methanol were added, and stirred at 20-25° C. for 3 hours. The reaction raw material 4b was consumed under LC monitoring. 10 ml of water was added and was stirred for 10 min. The mixture was partitioned, and the organic layer was washed twice with water, dried over anhydrous sodium sulfate, and concentrated to give compound 3b (2.56 g, yield: 85%).

¹H NMR (400 MHz, Chloroform) 8.27 (s, 1H), 8.09 (s, 1H), 7.99 (s, 1H), 7.90 (s, 1H), 7.62 (s, 1H), 7.33 (t, J=6.0 Hz, 6H), 5.46 (s, 1H), 5.20 (d, J=3.1 Hz, 3H), 5.02 (s, 2H), 4.49 (s, 1H), 3.35 (d, J=8.7 Hz, 4H), 3.21 (s, 1H), 3.12 (s, 1H), 2.83 (d, J=16.6 Hz, 2H), 2.75 (s, 1H), 2.54 (d, J=17.7 Hz, 1H), 2.34 (s, 1H), 1.88 (s, 1H).

### Example 7: Synthesis of Compound 3c

[chemical structure]

In a 100 ml reaction flask, 2.65 g of compound 4a, 1.74 g of pyridine tribromide compound, 26.5 ml of dichloromethane, 3.0 ml of methanol were added, and stirred at 20-25° C. for 3 hours. The reaction raw material 4a was consumed under LC monitoring. 5 ml of hydrobromic acid was added, and stirred for 30 min, then 10 ml of 10% KHCO₃ was added, and stirred for 10 min. The mixture was partitioned, and the organic layer was washed twice with water, dried over anhydrous sodium sulfate, and concentrated to give compound 3c (2.1 g, yield 83%).

¹H NMR (400 MHz, Chloroform) 8.27 (s, 1H), 8.09 (s, 1H), 7.99 (s, 1H), 7.90 (s, 1H), 7.62 (s, 1H), 7.35 (s, 1H), 5.50 (s, 1H), 5.30 (s, 1H), 4.78 (s, 1H), 3.55-3.27 (m, 3H), 3.14 (s, 1H), 2.83 (d, J=12.8 Hz, 1H), 2.75 (s, 1H), 2.57 (s, 1H), 2.47 (s, 1H), 2.35 (s, 1H), 2.10 (s, 1H), 1.48 (d, J=18.2 Hz, 1H).

### Example 8: Synthesis of Compound 2a

[chemical structure]

3.0 g of compound 3a, 1.7 g of compound 8, 0.55 g of potassium carbonate, and 15 ml of tetrahydrofuran were added into a 100 ml reaction flask, then stirred at 40-45° C. for 16 hours, and the reaction raw material 3a was consumed under LC monitoring. 30 ml ethyl acetate and 30 ml water were added, and stirred for 10 min. The organic phase was separated and the aqueous phase was extracted once again with 30 ml of ethyl acetate. The organic phases were combined, dried over sodium sulfate and concentrated to give compound 2a (3.85 g, yield 96.0%).

¹H NMR (400 MHz, Chloroform) δ 8.27 (s, 1H), 8.09 (s, 1H), 7.99 (s, 1H), 7.90 (s, 1H), 7.62 (s, 1H), 7.35 (s, 1H), 6.10 (d, J=27.4 Hz, 2H), 6.03 (s, 1H), 5.37 (s, 1H), 5.26 (d, J=18.2 Hz, 3H), 4.89 (s, 1H), 4.42 (s, 1H), 4.29 (s, 1H), 3.63 (s, 3H), 3.36-3.31 (m, 3H), 3.20 (s, 1H), 3.09 (s, 1H), 2.85 (s, 1H), 2.74 (d, J=8.0 Hz, 1H), 2.32 (s, 2H), 2.22 (d, J=12.2 Hz, 1H), 2.56-1.76 (m, 9H), 2.41-1.76 (m, 7H), 2.08-1.76 (m, 3H), 1.79-1.76 (m, 1H), 1.42 (s, 9H), 1.26 (s, 3H), 0.96 (s, 6H).

### Example 9: Synthesis of Compound 2b

[chemical structure]

In a 100 ml reaction flask, 3.0 g of compound 3b, 1.6 g of compound 8, 0.5 g of potassium carbonate, and 15 ml of tetrahydrofuran were added, and then stirred at 40-45° C. for 16 hours. The reaction raw material 3b was consumed under LC monitoring. 30 ml ethyl acetate and 30 ml of water were added and stirred for 10 min. The organic phase was separated, and the aqueous phase was extracted once again with 30 ml of ethyl acetate. The combined organic phase was dried over sodium sulfate and concentrated to give compound 2b (3.8 g, yield 96.0%).

¹H NMR (400 MHz, Chloroform) 8.27 (s, 1H), 8.09 (s, 1H), 7.99 (s, 1H), 7.90 (s, 1H), 7.62 (s, 1H), 7.33 (t, J=6.0 Hz, 6H), 6.11 (s, 1H), 6.19-5.52 (m, 3H), 5.26 (s, 2H), 5.05-4.93 (m, 4H), 4.46 (s, 1H), 4.32 (s, 1H), 3.63 (s, 3H), 3.35 (d, J=5.1 Hz, 4H), 3.22 (s, 1H), 3.10 (s, 1H), 2.85 (s, 1H), 2.74 (d, J=8.0 Hz, 1H), 2.53 (s, 1H), 2.45 (s, 1H), 2.39 (s, 1H), 2.29 (d, J=33.0 Hz, 2H), 2.21 (s, 1H), 2.07 (s, 1H), 1.92 (s, 1H), 1.82 (s, 1H), 1.26 (s, 3H), 0.96 (s, 6H).

### Example 10: Synthesis of Compound 2c

[chemical structure]

2.5 g of compound 3c, 1.7 g of compound 8, 0.55 g of potassium carbonate and 15 ml of tetrahydrofuran were added into a 100 ml reaction flask, then stirred at 40-45° C. for 16 hours, and the reaction raw material 3c was consumed under LC monitoring. 30 ml ethyl acetate and 30 ml water were added, and stirred for 10 min. The organic phase was separated and the aqueous phase was extracted once again with 30 ml of ethyl acetate. The combined organic phases was dried over sodium sulfate and concentrated to give compound 2c (3.16 g, yield 90.4%).

¹H NMR (400 MHz, Chloroform) δ 8.27 (s, 1H), 8.09 (s, 1H), 7.99 (s, 1H), 7.90 (s, 1H), 7.62 (s, 1H), 7.35 (s, 1H), 6.07 (s, 1H), 5.99 (s, 1H), 5.28 (d, J=6.6 Hz, 1H), 5.06 (s, 1H), 4.77 (s, 1H), 4.58 (s, 1H), 3.63 (s, 1H), 3.36-3.18 (m, 3H), 3.15 (s, 1H), 2.85 (s, 1H), 2.74 (d, J=8.0 Hz, 1H), 2.47 (dd, J=15.2, 10.0 Hz, 2H), 2.19 (d, J=13.0 Hz, 1H), 2.10 (d, J=0.5 Hz, 1H), 1.82 (s, 1H), 1.52 (s, 1H), 1.40 (s, 1H), 1.26 (s, 2H), 0.96 (s, 3H).

### Example 11: Synthesis of Compound 1a

[chemical structure]

In a 100 ml reaction flask, 1.65 g of compound 2a, 3.1 g of ammonium acetate, 3.0 ml of ethylene glycol methyl ether and 33 ml of toluene were added, and stirred at 90-95° C. for 16 hours. The reaction raw material 2a was consumed under LC monitoring. The reaction mixture was washed with 10 ml of hot water, then dried over anhydrous sodium sulfate, and evaporated to give compound 1a (1.36 g, yield: 85.0%).

### Example 12: Synthesis of Compound 1b

[chemical structure]

In a 100 ml reaction flask, 1.65 g of compound 2b, 3.0 g of ammonium acetate, 3.0 ml of ethylene glycol methyl ether, and 33 ml of toluene were added, and stirred at 90-95° C. for 16 hours. The reaction raw material 2b was consumed under LC monitoring. The reaction mixture was washed with 10 ml of hot water, then dried over anhydrous sodium sulfate, and evaporated to give compound 1b (1.38 g, yield: 86.8%).

¹H NMR (400 MHz, Chloroform) δ 8.27 (s, 1H), 8.09 (s, 1H), 8.00 (d, J=8.0 Hz, 2H), 7.48 (s, 1H), 7.33 (t, J=6.0 Hz, 6H), 6.33 (s, 1H), 5.30 (s, 2H), 5.04-4.92 (m, 4H), 4.80-4.72 (m, 1H), 4.69 (s, 1H), 4.80-4.31 (m, 3H), 3.63 (s, 3H), 3.35 (d, J=7.1 Hz, 4H), 3.22 (s, 1H), 3.06 (s, 1H), 2.98 (d, J=4.5 Hz, 4H), 2.73 (s, 1H), 2.22 (dt, J=49.4, 26.3 Hz, 6H), 2.18 (s, 1H), 2.27-1.85 (m, 4H), 2.06 (d, J=4.0 Hz, 1H), 1.82 (s, 1H), 1.26 (s, 3H), 0.96 (s, 6H).

### Example 13: Synthesis of Compound 1c

[chemical structure]

In a 100 ml reaction flask, 1.43 g of compound 2c, 3.1 g of ammonium acetate, 2.8 ml of ethylene glycol methyl ether and 28 ml of toluene were added, and stirred at 90-95° C. for 16 hours. The reaction raw material 2c was consumed under LC monitoring. The reaction mixture was washed with 10 ml of hot water, then dried over anhydrous sodium sulfate, and evaporated to give compound 1c (1.11 g, yield: 80.1%).

### Example 14A: Synthesis of Compound VLP-1

[chemical structure]

2.5 g of compound 1a, 7.5 g of active manganese dioxide, and 25 ml of dichloromethane were added into a reaction flask, and stirred at room temperature for 16 hours. The reaction raw material 1a was consumed by LC monitoring. 1.0 g of celite was added and stirred for 5 min, and suction-filtered to give a filtrate, which was concentrated to afford intermediate 1a-M.

Intermediate 1a-M was added to 8.7 ml of methanol and stirred to dissolve the intermediate. 6.3 ml of 3.0 M hydrogen chloride/methanol solution was added and stirred at 60° C. for 4H, cooled to 0° C., and the pH was adjusted to 7-8 with 25% sodium methoxide in methanol. The celite was added, and warmed to room temperature. The solids were removed by suction filtration, and washed with 5 ml of methanol. The filtrate was heated to 60° C., and 0.75 ml of 85% phosphoric acid was added. The mixture was aged for 4 hours, cooled to room temperature, and the compound VLP-1 phosphate was obtained by suction filtration.

1.0 g of compound VLP-1 phosphate was dissolved in 10 ml of water, 10 ml of dichloromethane was added, and 28% aqueous ammonia was added dropwise, stirred for 10 min, and allowed to stand for being layered. The upper aqueous phase was separated, and the organic phase was washed once, and dried over anhydrous sodium sulfate, and concentrated to give the compound VLP-1 (1.88 g, purity 98.70%, yield 86.2%).

### Example 14B: Synthesis of Compound VLP-1

[chemical structure]

2.5 g of compound 1b, 7.0 g of active manganese dioxide, and 25 ml of dichloromethane were added into a reaction flask, and stirred at room temperature for 16 hours. The reaction raw material 1b was consumed under LC monitoring. 1.0 g of celite was added, stirred for 5 min, and suction-filtered to give a filtrate, which was concentrated to afford intermediate 1b-M.

Intermediate 1b-M was added to 15 ml of methanol and stirred to dissolve the intermediate. 0.2 g of 5% Pd/C was added, stirred at room temperature for 16 hours, and Pd/C was removed by suction filtration.

Pd/C was washed with 5 ml of methanol. The combined filtrate was heated to 60° C., and 0.75 ml of 85% phosphoric acid was added. The mixture was aged for 4 hours, cooled to room temperature, and the compound VLP-1 phosphate was obtained by suction filtration.

1.0 g of compound VLP-1 phosphate was dissolved in 10 ml of water, 10 ml of dichloromethane was added, and 28% strong aqueous ammonia was added dropwise, stirred for 10 min, and allowed to stand foe being layered. The upper aqueous phase was separated, and the organic phase was washed once, and dried over anhydrous sodium sulfate. The mixture was concentrated to give the compound VLP-1 (1.79 g, yield 86.0%).

### Example 14C: Synthesis of Compound VLP-1

[chemical structure]

2.5 g of compound 1c, 8.6 g of active manganese dioxide, and 30 ml of dichloromethane were added into a 100 mL reaction flask, and stirred at room temperature for 16 hours. The reaction raw material 4 was consumed under LC monitoring. 1.0 g of celite was added and stirred for 5 min, and suction-filtered to give a filtrate, which was concentrated to afford crude compound VLP-1.

The crude VLP-1 was dissolved in 7.0 ml of methanol, heated to 60° C., and 0.6 ml of 85% phosphoric acid was added. The mixture was aged for 4 hours, cooled to room temperature, and the compound VLP-1 phosphate was obtained by suction filtration.

1.0 g of compound VLP-1 phosphate was dissolved in 10 ml of water, 10 ml of dichloromethane was added, and 28% aqueous ammonia was added dropwise. The mixture was stirred for 10 min, and allowed to stand for being layered. The upper aqueous phase was separated, and the organic phase was washed once, and dried over anhydrous sodium sulfate. The mixture was concentrated to give the compound VLP-1 (1.99 g, yield 80%).

### Example 15: Synthesis of Compound 1

[chemical structure]

3.0 g of compound VLP-1, 1.34 g of D-phenylglycine, 1.0 ml of N-methylmorpholine and 45 ml of dichloromethane were added into a 50 ml reaction flask, stirred for dissolving the reactants, cooled to 5-10° C., and 1.44 g 4-(4,6-dimethoxytriazine-2-yl)-4-methylmorpholine hydrochloride (DMTMM) was added, and stirred for 16 hours. The reaction raw material VLP-1 was consumed under LC monitoring, and the reaction was quenched by adding 10 ml of water. The aqueous layer was separated, washed successively with 10% KHCO₃ and water, dried over sodium sulfate, and concentrated to give compound VLP (3.44 g, purity 99.56%, yield 89.9%).

¹H NMR (400 MHz, Chloroform) δ 8.27 (s, 1H), 8.09 (s, 1H), 7.99 (s, 2H), 7.85 (s, 1H), 7.59 (d, J=12.0 Hz, 2H), 7.30 (dd, J=20.0, 12.0 Hz, 6H), 6.79 (s, 1H), 5.96 (s, 1H), 5.53 (s, 2H), 5.32 (s, 1H), 5.12 (s, 1H), 4.87 (s, 1H), 4.31 (d, J=10.8 Hz, 2H), 3.90 (s, 1H), 3.63 (s, 6H), 3.52 (s, 1H), 3.34 (s, 3H), 3.25-3.06 (m, 1H), 2.73 (s, 1H), 2.45 (s, 1H), 2.38 (d, J=13.6 Hz, 2H), 2.19 (s, 1H), 2.07 (s, 1H), 1.94 (s, 1H), 1.82 (s, 1H), 1.26 (s, 3H), 0.96 (s, 6H).

All literatures mentioned in the present application are incorporated herein by reference, as though each one is individually incorporated by reference. Additionally, it should be understood that after reading the above teachings, those skilled in the art can make various changes and modifications to the present invention. These equivalents also fall within the scope defined by the appended claims.

1. A method for preparing an intermediate compound of velpatasvir represented by formula I, [chemical structure]
wherein it comprises the steps:
(1) condensing a compound of formula 7 and a compound of formula 6 to provide a compound of formula 5; [chemical structure]
(2) subjecting the compound of formula 5 to a cyclization reaction to prepare a compound of formula 4; [chemical structure]
(3) reacting the compound of formula 4 with a substitution reagent to prepare a compound of formula 3; [chemical structure]
(4) condensing the compound of formula 3 with a compound of formula 8 to obtain a compound of formula 2; [chemical structure]
(5) subjecting the compound of formula 2 to a cyclization reaction to give a compound of formula 1; [chemical structure]
in the above formulas,
Y is selected from the group consisting of —F, —Cl, —Br, —I, —OTs, —OSO₂CF₃, —SO₂R₆, —OP(O)(OR₆)₂; wherein R₆ is C1-C10 alkane group, C6-C10 aromatic hydrocarbon group or substituted aromatic hydrocarbon group; preferably, said Y is selected from the group consisting of: —Cl, —Br, —I;
Z is selected from the group consisting of H, alkali metal ions, alkaline earth metal ions; preferably, said Z is selected from the group consisting of: —H, —Na, —K, —Li, —Cs;
R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
R₂ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl, alkoxymethyl, alkylsulfonyl, substituted arylsulfonyl, L-methoxycarbonylvalyl;
wherein said “substituted” means that a group is substituted by one or more substituents selected from the group consisting of halogen, C₁-C₆ alkyl, C₂-C₁₀ acyl.

2. The method of claim 1, wherein in the step (2) and/or the step (5), the cyclization reaction is carried out in the presence of ammonia or a derivative thereof selected from the group consisting of C1-C6 ammonium carboxylate, ammonium salt of inorganic acid, carbamide, NH₃, methylsilicone diamine, or the combinations thereof, preferably ammonium acetate, ammonium formate, ammonium hydrogencarbonate, or the combinations thereof.

3. The method of claim 1, wherein in the step (4), the alkaline condition is provided by adding an alkaline agent selected from the group consisting of alkali metal hydrogencarbonate, alkaline earth metal hydrogencarbonate, alkali metal carbonate, alkaline earth metal carbonate, alkali metal hydroxide, alkaline earth metal hydroxide, alkali metal phosphate, alkaline earth metal phosphate, alkali metal hydrogen phosphate salt, alkaline earth metal hydrogen phosphate salt, DBU, DBN, [chemical structure]
or a combination thereof, wherein R₃, R₄, R₅ are each independently H or a C1-C4 alkane; preferably, the alkaline agent is selected from the group consisting of potassium carbonate, cesium carbonate, or the combination thereof.

4. The method of claim 1, wherein in the step (1), the alkaline agent includes, but is not limited to, an alkali metal or alkaline earth metal hydrogencarbonate, alkali metal or an alkaline earth carbonate, alkali metal or alkaline earth metal hydroxide, alkali metal or alkaline earth metal hosphate, alkali metal or alkaline earth metal hydrogen phosphate, DBU, DBN, [chemical structure]
wherein R₃, R₄, and R₅ are each independently H or C1-C4 alkane; preferably potassium carbonate, cesium carbonate.

5. The method of claim 1, wherein in the step (3), the substitution reagent is halogenating reagent, preferably halogenating reagent selected from the group consisting of N-halogenated succinimide, 5,5-dimethyl-1,3 dihalohydantoin, halogen, chlorinated halide, lithium halide, sodium halide, potassium halide, pyridine trihalide, trihalide tetrabutyl ammonium, trihalide trimethylammonium, trihalide triethylammonium, sodium hypohalite, potassium hypohalite, lithium hypohalite, sodium sulfite, potassium sulfite, sodium sulfite, or a combination thereof, preferred is N-bromosuccinimide (NBS), 5,5-dimethyl-1,3-dibromohydantoin, pyridine tribromide, or the combinations thereof.

6. An intermediate compound of the following formula: [chemical structure]
wherein, R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
X is selected from the group consistin of —H, —F, —Cl, —Br, —I, —OTs, —OSO₂CF₃, —SO₂R₆, —OP(O)(OR₆)₂, [chemical structure]
wherein R is C1-C10 alkyl, C6-C10 aromatic hydrocarbon group or substituted aromatic hydrocarbon group;
preferably, the compound is selected from the group consisting of: [chemical structure]
wherein, Y is selected from the group consisting of —F, —Cl, —Br, —I, —OTs, —OSO₂CF₃, —SO₂R₆, —OP(O)(OR₆)₂; wherein R is C1-C10 alkyl, C6-C10 aromatic hydrocarbon group or substituted aromatic hydrocarbon group.

7. An intermediate compound of the following formula:

[chemical structure]

8. A method for preparing an intermediate compound of velpatasvir represented by formula I, comprising steps:
subjecting a compound of formula 2 to a cyclization reaction to give a compound of formula 1; [chemical structure]
in the above formulas,
R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
R₂ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl, alkoxymethyl, alkylsulfonyl, substituted arylsulfonyl, L-methoxycarbonylvalyl;
wherein said “substituted” means that a group is substituted by one or more substituents selected from the group consisting of halogen, C1-C6 alkyl, C2-C10 acyl.

9. The method of claim 8, wherein the method comprises the following steps:
Under an alkali condition, condensing the compound of formula 3 with compound of formula 8 to obtain a compound of formula 2; [chemical structure]
wherein,
Y is selected from the group consisting of —F, —Cl, —Br, —I, —OTs, —OSO₂CF₃, —SO₂R₆, —OP(O)(OR₆)₂; wherein R₆ is C1-C10 alkane group, C6-C10 aromatic hydrocarbon group or substituted aromatic hydrocarbon group; preferably, said Y is selected from the group consisting of: —Cl, —Br, —I;
Z is selected from the group consisting of H, alkali metal ions, alkaline earth metal ions; preferably, said Z is selected from the group consisting of: —H, —Na, —K, —Li, —Cs.

10. A method of preparing velpatasvir, wherein the method comprises the steps:
Subjecting a compound of formula 2 to a cyclization reaction to give a compound of formula 1; [chemical structure]
and
preparing verapitavir with the compound of formula 1;
in the above formulas,
R₁ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl (Fmoc), alkoxymethyl, alkylsulfonyl, substituted arylsulfonyl, D-phenylglycyl;
R₂ is selected from the group consisting of hydrogen, organosilicon derivative protecting groups, alkoxycarbonyl, cycloalkoxycarbonyl, cycloalkanoyl, substituted alkanoyl, substituted aroyl, benzyl, benzyloxycarbonyl (Cbz), 9-fluorenylmethoxycarbonyl, alkoxymethyl, alkylsulfonyl, substituted arylsulfonyl, L-methoxycarbonylvalyl;
wherein said “substituted” means that a group is substituted by one or more substituents selected from the group consisting of halogen, C1-C6 alkyl, C2-C10 acyl.
